(19) United States
(12) Patent Application Publication
(10) Pub. No.: US 2013/0018259 A1
(43) Pub. Date: Jan. 17, 2013

(54) Target Identification Tool For Intra-Lumenal Localization

(71) Applicants: Thomas Borillo, Medina, MN (US); Jerone Kent Grudem, JR., Rogers, MN (US)

(72) Inventors: Thomas Borillo, Medina, MN (US); Jerone Kent Grudem, JR., Rogers, MN (US)

(73) Assignee: SuperDimension, Ltd.

(21) Appl. No.: 13/536,802

(22) Filed: Jun. 28, 2012

Related U.S. Application Data
(60) Provisional application No. 61/501,931, filed on Jun. 28, 2011.

(51) Int. Cl. A61B 6/00

(52) U.S. Cl. 600/433

(57) ABSTRACT

An intralumenal marker that includes an atraumatic anchoring portion that allows the marker to be fixed within a body lumen, such as an airway, without risk of migration. The anchoring portion may be an elongate body that reassumes a relaxed, coiled configuration after being delivered from a catheter. A body portion is attached to the anchoring portion and provides a function such as radiopaque marking, drug delivery, illumination, or the like.

### RELATED APPLICATIONS

This application claims priority to U.S. Provisional Application Ser. No. 61/501,931 filed Jun. 28, 2011 entitled Target Identification Tool For Intra-Lumenal Localization, which is hereby incorporated herein by reference in its entirety.

### BACKGROUND OF THE INVENTION

In the case of a suspected lung mass in a high risk patient for lung cancer, it is the current standard of care to send the patient for radical removal of the mass. Certain portions of these surgeries are made by Video Assisted Thoracotomy Surgery (VATS), which is a minimally invasive surgery, and invasive Thoracic Surgery. Obtaining accurate diagnosis in the least invasive means possible as quickly as possible is essential. During VATS, it is often very hard to recognize the suspected small lung masses during the procedure. VATS success is limited by the ability to visualize and palpate the nodule if it is less than 10 mm in size and if it is more than 5 mm from a pleural surface. Historically, in 63% to 82% of cases there is an inability to visualize or palpate a detected nodule. (1. Burdine, et al. CHEST 2002; 122:1467, 2. Suzuki, et al. CHEST 1999; 115:563). Minimally invasive surgery is becoming more and more popular and holds similar challenges to those seen in VATS when used in the abdominal cavity, the urogenital system or other parts of the body.

A lung mass (solitary pulmonary nodules (SPN) or other) in the periphery of the lungs that is identified by X-ray machine or CT must also be physically identified by the surgeon for removal. However, visual identification of the mass may often be difficult due to tissue obstructions, such as, when the nodule is buried deep in the lung tissue.

Lack of visual identification creates problems. In some instances, surgeons discover lesions during surgery that were not earlier identified by a referring physician or radiologist. In this case, the surgeon needs to decide which of the lesions is suspected to be cancerous. Therefore, to avoid mistakes, the surgeon typically removes a larger portion of the tissue, ensuring the entire lesion is removed but also increasing tissue trauma, the possibility of complications, patient suffering, and so forth. In other cases, lack of visual identification results in the excision of healthy tissue rather than the targeted lesion.

In other body cavities similar challenges are encountered since visibility and the means to identify specific pre-planned lesions as were identified by medical imaging is often limited.

Most current methods for identifying masses and other such lesions and tissues may best be characterized as “from the outside to the inside,” and are often rather complex, invasive and risky. Such methods include, for example, manual identification (e.g., finger palpation through the rib cage), intrathorascopic ultrasound, transthoracic placement of an external wire, injecting solidifying liquids, dye injection, TC-99 injection, radiopaque markers such as barium or injectable coils, guidance by CT, intrathorascopic ultrasound, fluoroscopy-assisted thoracoscopic resection, etc.

There are current challenges with external beam radiation delivery due to the inability to see the tumor during treatment. Accurate alignment of sterotactic planning onto the patient, before the procedure, is required for accurate real-time tracking of the tumor. Additionally, tumor position in the lungs is changing as a result of the normal respiratory cycle, unpredictable baseline shifts and variable amplitude of respiratory rates. Consequently, an insufficient dose of radiation may be delivered due to its toxic effects on surrounding healthy lung tissue and may lead to failure to control tumor growth. Because of these challenges, fiducial markers are often used in soft tissue to guide focused-beam radiation treatment.

One of the major drawbacks to fiducial marker placement is delivery of the marker transthoracically. This approach can lead to pneumothorax or collapsed lungs because often the patients already have compromised lung function. In addition to the risk of pneumothorax there is also the complication of marker migration. Unlike the relatively static, homogeneous tissue of the prostate, the lung tissue moves significantly with the breathing cycle and is also porous and interlaced with airways. As a result, an implanted seed is prone to migrate, typically out of the channel formed during placement, and fall down an airway. Once in the airway, the seed will either settle in a distal portion of the lungs, or be coughed out.

If an inert or active marker seed or temporary catheter migrates, the target is lost. If the therapy vehicle is expectorated, the treatment ends prematurely. Even worse, if the delivery vehicle migrates away from the target, therapy is administered to healthy tissue instead of a tumor, thereby damaging the healthy tissue and sparing the tumor.

In many cases, it is desired to mark a location inside a lumen, such as the airway, rather than inside tissue adjacent an airway. Placing a marker within the airway is particularly difficult. The walls of the airways expand and contract with every breath, are filled with a moving medium which, during events like coughing and sneezing, can become violently forceful, and are lined with mucous. Additionally, in order to prevent migration and/or restriction of the airway, which can result in coughing or collapsing of the lung downstream of the marker location, the marking device must have a profile that is non-restrictive, while still presenting a bright imaging profile.

There is a need for an improved identification device or marking device and method of introducing this device into the body. More specifically, there is a need for an identification device or marking device that can be placed within the airway without migration. Preferably, the marker device would have minimal impact on the tissue of the airways.

### SUMMARY OF THE INVENTION

In view of the foregoing, one aspect of the present invention is to provide an identification or marking device and method that overcomes the limitations of the prior art.

Another aspect of the present invention is to provide an identification or therapeutic device that may be placed permanently or semi-permanently (removable only with excision of the surrounding tissue) or removably (without significant trauma to the surrounding tissue).

Another aspect of the present invention is to provide an identification or therapeutic device that may be pre-, intra- or post operatively activated and implanted in the location of interest or adjacent to the location of interest within the body (for example, at or near a mass and surrounding tissues desired for extraction).

Yet another aspect of the invention provides a marking device that may be securely placed within the airways, with minimal risk of migration.

Another aspect of the invention provides a marking device that may be securely placed within the proximal airways without traumatizing the airway tissue.

Another aspect of the invention provides a marking device that may be securely placed within the distal airways that promotes a healing response by the airway tissue, which then secures the device in place.

Still another aspect of the invention provides a marking device that may be securely placed within the airways, has a bright imaging profile, but does not impede airflow significantly.

### BRIEF DESCRIPTION OF THE DRAWINGS

FIG. 1 is a perspective view of an embodiment of the device of the present invention;

FIG. 2 is a perspective view of the embodiment of the device of the present invention;

FIG. 3a is a perspective view of an embodiment of the device of the present invention in an unconstrained state;

FIG. 3b is a perspective view of the device of FIG. 3a constrained within an airway;

FIG. 4a is a perspective view of an embodiment of the device of the present invention in an unconstrained state;

FIG. 4b is a perspective view of the device of FIG. 4a constrained within an airway;

FIG. 5a is an end view of an embodiment of the device of the present invention;

FIG. 5b is a profile view of the device of FIG. 5a;

FIG. 6a is an end view of an embodiment of the device of the present invention;

FIG. 6b is a profile view of the device of FIG. 6a;

FIG. 7a is an end view of an embodiment of the device of the present invention; and,

FIG. 7b is a profile view of the device of FIG. 7a.

### DETAILED DESCRIPTION OF THE INVENTION

In general, the present invention includes an identification or therapeutic device comprising a body portion and an anchoring portion, which is introducible into an intra-body structure (e.g., a mass or lesion) and/or an anatomical space to mark a location of interest (e.g., a tissue layer and/or lumen of a body cavity). The identification device of the present invention may include a power source, either external to the body or internally at or near the body portion or some combination thereof. It is understood that any of the various anchoring portions described below may be used with any of the body portions. It is also understood that the body portions may give off energy, such as light energy (i.e. glow-in-the-dark materials, LEDs, incandescent devices, etc.), thermal energy, radiation, RF energy, acoustic energy, or cryoenergy.

Furthermore, the various embodiments of the body portions may be constructed of various application-specific materials. For example, the body portions may be loaded with chemicals or dyes that enhance localization. Non-limiting examples include: BaSO4, bismuth, copper, gold, and platinum. Also, the body portions could be loaded with drugs and/or chemotherapy agents for treatment and have features such as controlled elution and diffusion rates. Non-limiting examples of these agents include antineoplastics, antiobiotics and others.

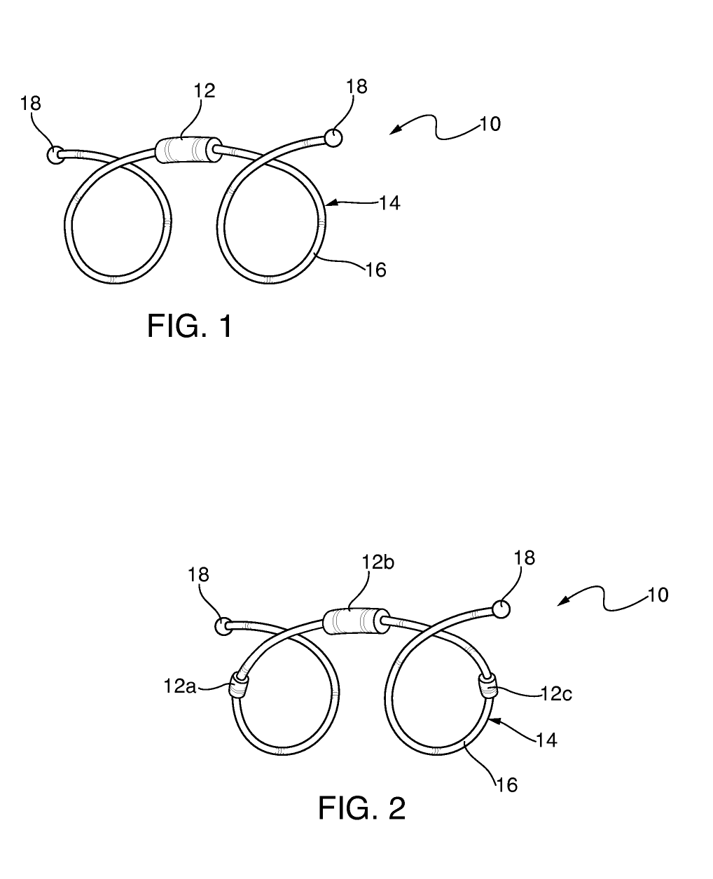

One embodiment of the present invention is shown in FIG. 1, which illustrates an identification or therapeutic device 10, including a body portion 12 and anchoring portion 14. The body portion 12 may be any energy source or simply a marker or a focusing element for RF energy, as described above. If an energy source is used, it is understood that appropriate additional equipment will be used in order to receive and identify the energy being transmitted. One embodiment provides a body portion 12 that is highly radiopaque, such as gold. Due to the high radiopacity of gold, the gold body portion 12 may be sized small enough that it poses little to no restriction on airflow, while still being highly visible by an imaging device, such as CT or fluoroscopy.

The embodiment shown in FIG. 2 includes a similar anchoring portion 14 with multiple body portions 12a, 12b, 12c, and so on. Providing multiple body portions provides a bright imaging profile while still maintaining a low resistance to airflow. Additionally, multiple body portions provide information during imaging as to the orientation of the device 10. For example, if the alignment of the body portions 12 changes relative to each other over time, it may be indicative that migration is occurring. Information may also be gleaned as to whether the airways distal of the body portion are remaining open. If the body portions can be seen moving rhythmically with inhalation and exhalation, it is indicative that air is flowing past the body portions 12. However, if the body portions 12 are not moving despite inhalation and exhalation, it may indicate that air is not flowing past the body portions.

Conversely, the embodiment shown in FIG. 1 includes a single body portion 12. The single body portion design allows a larger quantity of radiopaque material, such as gold, to be concentrated in a single location without impeding airflow. A single mass of gold provides a brighter single point in imaging than the smaller body portions used in the multiple body portion embodiments. It is noted that all of the various anchor designs shown in the Figures and described herein, may be used with a single body portion 12 or multiple body portions 12a, 12b, 12c, etc.

The anchoring portion 14 is constructed and arranged to be placed into a small delivery catheter and to expand upon exit from the catheter into a shape that secures the device 10 within an airway. Several examples are shown in the figures. The anchoring portion 14 shown in FIGS. 1 and 2 comprises a coil 16 that is preferably constructed of a material, such as Nitinol, that is biocompatible and has shape memory qualities. The anchoring portion 14 may also include blunt end caps 18 that prevent then ends from penetrating the lung tissue during delivery, and also ensure that the device will be able to slide through the delivery catheter. The anchoring portion 14 is capable of being straightened and contained in a delivery catheter for extended periods while still reassuming a deployed shape when released from the catheter. The anchoring portion shown in FIGS. 1 and 2 has a spiral or coil shape when released. This coil shape places a gentle outward force on the airways, and also expands and contracts with the airways to maintain the desired, implanted location.

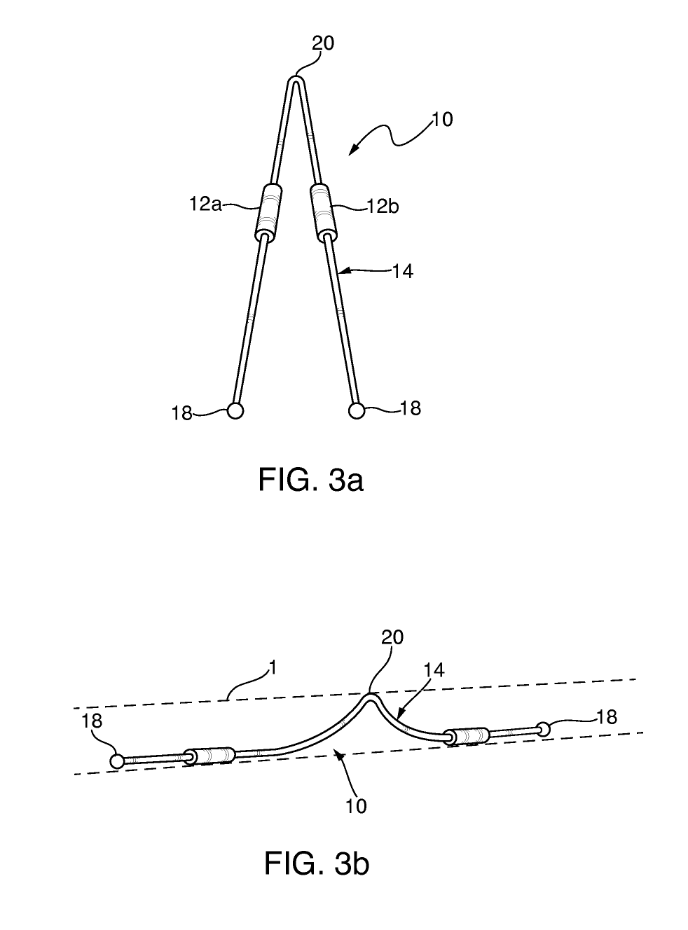

FIGS. 3a and 3b show an anchoring portion 14 that is suitable for implantation in a large airway. The anchoring portion 14 includes a single bend 20 that can be as much as 180 degrees when unconstrained. FIG. 3a shown the device 10 in an unconstrained configuration. FIG. 3b shows the device 10 in a deployed configuration within an airway 1, shown in phantom lines.

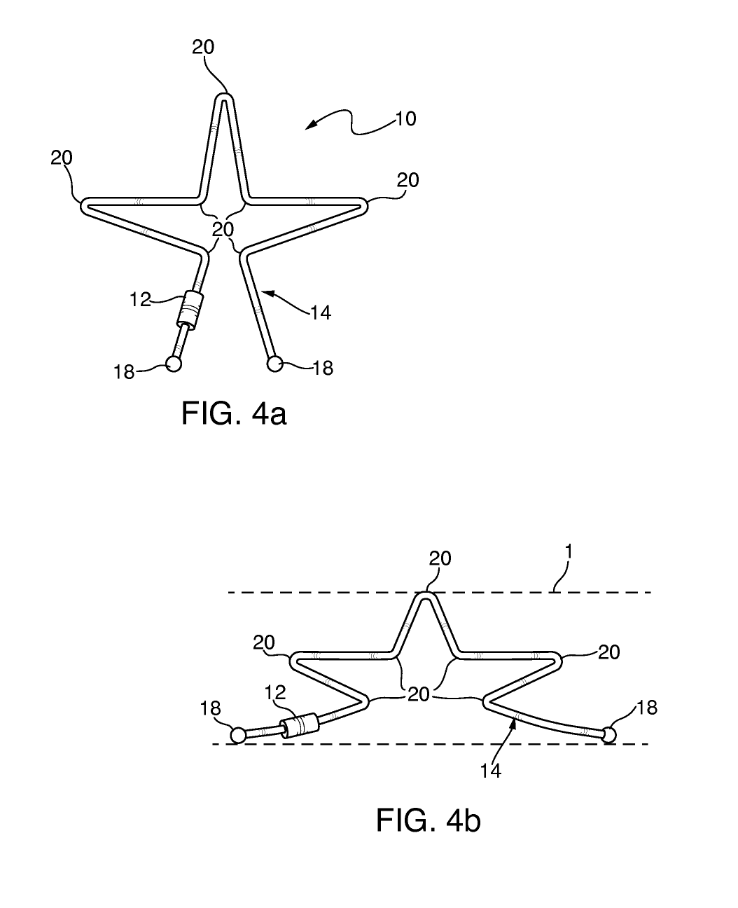

FIGS. 4a and 4b also show an anchoring portion 14 that is constructed of a resilient material, such as a memory metal, that can be elongated and placed into a small delivery catheter. Upon release from the catheter, the anchoring portion 14 expands to form a star shape having a plurality of points 20. FIG. 4a shows the device 10 in an unconstrained configuration. FIG. 4b shows the device 10 within an airway 1. Like the coil shape, the star shape places gentle outward force on the airways, and expands and contracts with the airways to maintain the desired, implanted location. However, the star shape also expands and contracts in such a manner that the contact points of the star, those points 16 contacting tissue, do not slide as a result of expansion and contraction. Hence, wear on the tissue contacted by the device is minimized.

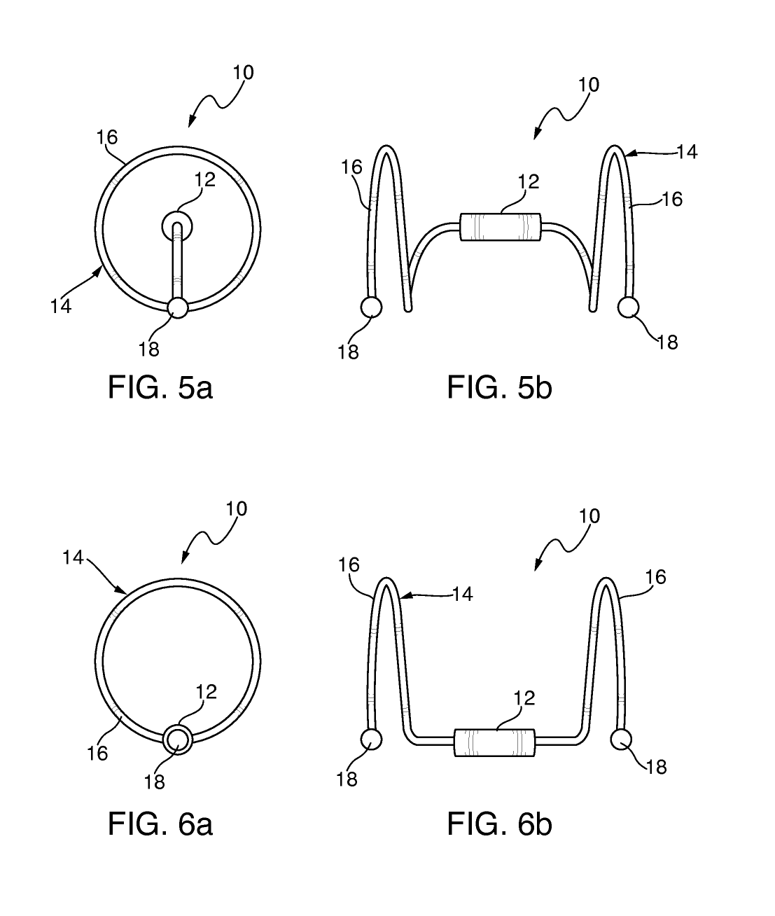

FIGS. 5a and 5b show a device 10 having an anchoring portion 14 that is constructed and arranged with coils 16 at either end of the device and a center portion 22 that extends between the two coils 16. The center portion 22 holds the body portion 12 in the center of the airway. Like the other embodiments, the ends of the anchoring device include blunt end caps 18.

FIGS. 6a and 6b show a device 10 having an anchoring portion 14 that is constructed and arranged with coils 16 at either end of the device and a center portion 22 that extends between the two coils 16. The center portion 24 holds the body portion 12 along a wall of the airway. Like the other embodiments, the ends of the anchoring device include blunt end caps 18. This embodiment maximizes the amount of airflow allowed to continue to flow through the airway.

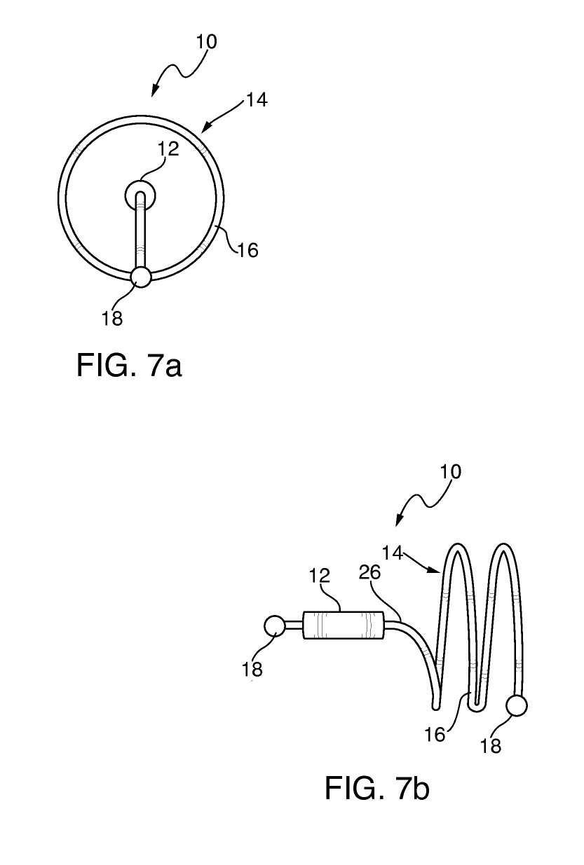

FIGS. 7a and 7b show a device 10 having an anchoring portion 14 that is constructed and arranged with a coil 16 at one end of the device and an axial portion 26 the other end of the device. The axial portion 26 holds the body portion 12 in the center of the airway. Because the axial portion 26 is at the end of the device 10, a user may find it easier to predictably place the body portion in a desired location. Like the other embodiments, the ends of the anchoring device include blunt end caps 18.

The method of the present invention is thus described as a method of marking a location within a lumen of a patient that includes placing an intralumenal marker, having an anchoring portion and a body portion attached to said anchoring portion, within a catheter in an elongated configuration; navigating said catheter to a target location within a lumen of a patient; deploying said intralumenal marker from said catheter into said lumen; and allowing said anchoring portion of said marker to expand within said lumen, thereby placing atraumatic pressure on walls of said lumen such that said body portion is fixed within said lumen.

The step of allowing said anchoring portion of the marker to expand within the lumen, thereby placing atraumatic pressure on walls of the lumen such that the body portion is fixed within the lumen, can include allowing the anchoring portion of the marker to expand within the lumen, thereby placing atraumatic pressure on walls of the lumen such that the body portion is axially centered within the lumen.

The step of allowing the anchoring portion of the marker to expand within the lumen, thereby placing atraumatic pressure on walls of the lumen such that the body portion is fixed within the lumen, can include allowing the anchoring portion of the marker to expand within the lumen, thereby placing atraumatic pressure on walls of the lumen such that the body portion is adjacent a sidewall of the lumen.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

We claim:

1. An intralumenal marker comprising:
an atraumatic anchoring portion having a first configuration and a second configuration, said first configuration elongated to facilitate deployment via a catheter, said second configuration comprising a relaxed state having a size greater than a diameter of an intended targeted body lumen such that when implanted, said anchoring portion places gentle outward pressure on walls of said body lumen, thereby preventing migration of said marker;
at least one body portion attached to said anchoring portion, said at least one body portion comprising a material selected from the group consisting of localization-enhancing materials, drugs, chemotherapy agents, and energy-emitting materials.

2. The intralumenal marker of claim 1 wherein said relaxed state comprises a spiral.

3. The intralumenal marker of claim 1 wherein said relaxed state comprises a coil.

4. The intralumenal marker of claim 1 wherein said relaxed state comprises a single bend.

5. The intralumenal marker of claim 1 wherein said relaxed state comprises a star-shape.

6. The intralumenal marker of claim 1 wherein said atraumatic anchoring portion comprises blunt ends.

7. The intralumenal marker of claim 1 wherein one of said at least one body portions is attached to said anchoring portion such that when said marker is implanted in a lumen, said one of said at least one said body portions is located axially in said lumen.

8. The intralumenal marker of claim 1 wherein said at least one body portion is attached to said anchoring portion such that when said marker is implanted in a lumen, said body portion is located along a sidewall of said lumen.

9. The intralumenal marker of claim 1 wherein said at least one body portion comprises a localization-enhancing material selected from the group consisting of BaSo4, bismuth, copper, gold and platinum.

10. The intralumenal marker of claim 1 wherein said at least one body portion comprises a material selected from the group consisting of antineoplastic and antibiotics.

11. The intralumenal marker of claim 1 wherein said at least one body portion comprises a controlled elution rate.

12. The intralumenal marker of claim 1 wherein said at least one body portion comprises a controlled diffusion rate.

13. The intralumenal marker of claim 1 wherein said at least one body portion comprises a single body portion.

14. The intralumenal marker of claim 1 wherein said at least one body portion comprises a plurality of body portions spaced apart along said anchoring portion.

15. The intralumenal marker of claim 1 wherein said anchoring portion comprises Nitinol.

16. A method of marking a location within a lumen of a patient comprising:
placing an intralumenal marker, having an anchoring portion and a body portion attached to said anchoring portion, within a catheter in an elongated configuration;
navigating said catheter to a target location within a lumen of a patient;
deploying said intralumenal marker from said catheter into said lumen;
allowing said anchoring portion of said marker to expand within said lumen, thereby placing atraumatic pressure on walls of said lumen such that said body portion is fixed within said lumen.

17. The method of claim 16 wherein allowing said anchoring portion of said marker to expand within said lumen, thereby placing atraumatic pressure on walls of said lumen such that said body portion is fixed within said lumen comprises allowing said anchoring portion of said marker to expand within said lumen, thereby placing atraumatic pressure on walls of said lumen such that said body portion is axially centered within said lumen.

18. The method of claim 16 wherein allowing said anchoring portion of said marker to expand within said lumen, thereby placing atraumatic pressure on walls of said lumen such that said body portion is fixed within said lumen comprises allowing said anchoring portion of said marker to expand within said lumen, thereby placing atraumatic pressure on walls of said lumen such that said body portion is adjacent a sidewall of said lumen.

19. An intralumenal marker comprising:
an elongate anchoring portion having a straightened configuration and a coiled configuration, said straightened configuration facilitating deployment via a catheter, said coiled configuration comprising a relaxed state having a diameter greater than a diameter of an intended targeted body lumen such that when implanted, said anchoring portion places gentle outward pressure on walls of said body lumen, thereby preventing migration of said marker;
at least one body portion attached to said anchoring portion, said at least one body portion comprising a material selected from the group consisting of localization-enhancing materials, drugs, chemotherapy agents, and energy-emitting materials.

20. The intralumenal marker of claim 19 wherein said anchoring portion further includes an axial portion to which said body portion is attached such that said body portion is aligned within a targeted lumen when deployed.
